(19) United States
(12) Patent Application Publication
(10) Pub. No.: US 2025/0169730 A1
(43) Pub. Date: May 29, 2025

(54) SAMPLE COLLECTION DEVICE AND SAMPLE PROCESSING DEVICE

(71) Applicant: CANON MEDICAL SYSTEMS CORPORATION, Otawara-shi (JP)

(72) Inventors: Sayo KATSUKI, Utsunomiya (JP); Satoshi YAMANE, Nasushiobara (JP); Asuka HIRANO, Nasushiobara (JP)

(73) Assignee: CANON MEDICAL SYSTEMS CORPORATION, Otawara-shi (JP)

(21) Appl. No.: 18/963,914

(22) Filed: Nov. 29, 2024

(30) Foreign Application Priority Data
Nov. 29, 2023 (JP) 2023-201677

(51) Int. Cl. A61B 5/15

(52) U.S. Cl. CPC A61B 5/150343; A61B 2560/0425

(57) ABSTRACT

A sample collection device includes a sample collection unit. The sample collection unit has a contact surface, a retaining space, and an outer surface. The contact surface is capable of being in contact with a surface having a sample outflow hole of a subject. The retaining space has an opening on the contact surface. In the sample collection device, the retaining space stores a predetermined amount of the sample due to the surface tension of the sample, the predetermined amount of the sample being obtained from the sample flowing out of the sample outflow hole when the contact surface is pressed on the surface around the sample outflow hole.

### CROSS REFERENCE TO RELATED APPLICATIONS

This application is based upon and claims the benefit of priority from the prior Japanese Patent Application No. 2023-201677, filed on Nov. 29, 2023, the entire contents of which are incorporated herein by reference.

### FIELD

One or more embodiments described herein and illustrated in the drawings relate to a sample collection device and a sample processing device.

### BACKGROUND

It has recently been widely performed to collect a subtle amount of sample from punctuated skin of a subject such as a patient, to analyze the sample by an analysis device or the like, and to monitor biological information of the subject based on the information obtained from the analysis. The information obtained from the sample may also be used for the diagnosis of various kinds of diseases. The sample may be, for example, a fluid sample such as blood or pus. Conventionally, such an inspection has been performed by making a small hole on the skin with a disposable needle called “lancet” to squeeze the sample out of the hole, and collecting a predetermined amount of the sample with a micropipette or a narrow plastic or glass tube called “capillary.”

During a sample inspection in point of care testing (POCT), it is needed to collect a certain amount of inspection sample. Since the use of pipetman, which is generally used, requires technical expertise, a specialist is needed to perform the sample inspection. Furthermore, there is a problem in that the amount flowing out of the hole and the collected amount considerably fluctuate depending on the components and the viscosity of the sample. This problem makes the quantitative precision unstable.

There may be a case where a capillary is used. The sample collection using a capillary does not need a special technique, and anyone may collect the sample. However, since the capillary made of glass is easily broken, the use of capillary in a medical site may cause a risk. Furthermore, since it is more difficult to control the amount of sample collected by a capillary than the amount of sample collected by a pipetman, the quantitative precision is lower when a capillary is used. Thus, the use of a capillary is insufficient in the field of sample collection in POCT, since the sample collection in POCT would require a higher precision in the future.

A predetermined amount of sample may also be collected by weight measurement using a precision balance. However, precision balances are not suitable for the use in POCT since they are likely to be affected by environmental factors (vibrations, temperature, humidity, etc.).

In a sample inspection in POCT, it is intended to use a subtle amount of sample obtained by pricking the fingertip, for example, with a puncture device such as a lancet or a blood collection needle. In order to have a sufficient amount of sample, it is needed to press a portion around the punctuated hole to squeeze the sample. A conventional collection device requires three steps to collect a sample, including pressing a surrounding portion of a position to be punctured, collecting the sample, and processing the sample including diluting the sample. Performing such three steps would need a long time. If a long time passes during the collection and the processing of the sample, the sample (such as blood) may partially coagulate, which decreases the quantitative precision. Thus, it is required to shorten the time until the processing of the sample in POCT that requires a prompt inspection.

One of the problems to be solved by the embodiments described herein and illustrated in the accompanied drawings is to easily collect a certain amount of sample from a subject. The problems to be solved by the embodiments described herein and illustrated in the accompanied drawings are not limited to the above-described problem. There may be other problems such as those corresponding to the advantages or effects of the embodiments described below.

### BRIEF DESCRIPTION OF THE DRAWINGS

FIG. 1 is a perspective view illustrating an example of a sample collection device according to an embodiment.

FIG. 2 is a longitudinal cross section of the sample collection device shown in FIG. 1.

FIG. 3 is an explanatory diagram used for explaining a method of collecting a sample using the sample collection device.

FIG. 4 is an explanatory diagram used for explaining the method of collecting the sample using the sample collection device.

FIG. 5 is an explanatory diagram used for explaining the method of collecting the sample using the sample collection device.

FIG. 6 is an explanatory diagram used for explaining the method of collecting the sample using the sample collection device.

FIG. 7 is an explanatory diagram used for explaining the method of collecting the sample using the sample collection device.

FIG. 8 is a perspective view of a modification of the sample collection device.

FIG. 9 is a longitudinal cross section of the sample collection device shown in FIG. 8.

FIG. 10 is a perspective view of another modification of the sample collection device.

FIG. 11 is a perspective view of a further modification of the sample collection device.

FIG. 12 is a longitudinal cross section of the sample collection device shown in FIG. 11.

FIG. 13 is a diagram showing the sample collection device of FIG. 11 from the contact surface side.

FIG. 14 is a perspective view illustrating a still further modification of the sample collection device.

FIG. 15 is a perspective view illustrating a still further modification of the sample collection device.

FIG. 16 is a perspective view illustrating a still further modification of the sample collection device.

FIG. 17 is a perspective view illustrating a still further modification of the sample collection device.

FIG. 18 is a perspective view illustrating a still further modification of the sample collection device.

FIG. 19 is a perspective view illustrating a still further modification of the sample collection device.

FIG. 20 is a perspective view illustrating a still further modification of the sample collection device.

FIG. 21 is a perspective view illustrating a still further modification of the sample collection device.

FIG. 22 is a perspective view illustrating a still further modification of the sample collection device.

FIG. 23 is a perspective view illustrating a still further modification of the sample collection device.

FIG. 24 is a longitudinal cross section of a sample processing device including a sample collection device and a sample processing container.

FIG. 25 is a photograph of a sample collection device used in a first experiment.

FIG. 26 shows an evaluation of quantitative precision of the sample collected by using the sample collection device shown in FIG. 25.

FIG. 27 is a photograph of a sample collection device used in a second experiment.

FIG. 28 shows an evaluation of quantitative precision of the sample quantitative precision of the sample collected in the second experiment.

### DETAILED DESCRIPTION

Embodiments will now be described with reference to the accompanying drawings. The scaling and the ratio between dimensions of an element may be different from those of the actual element in each drawing, for the easy understanding and the convenience of illustration.

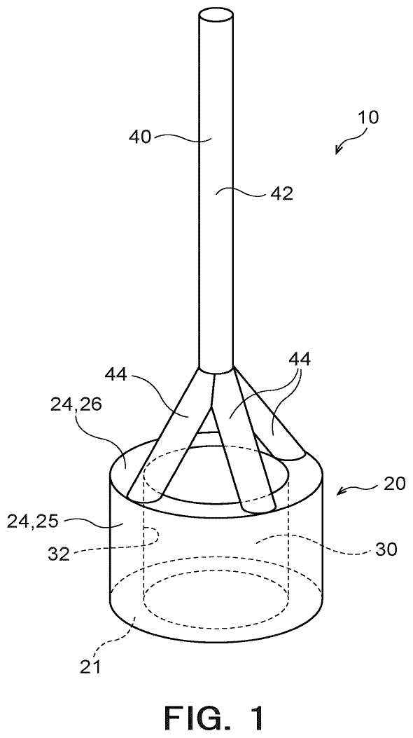

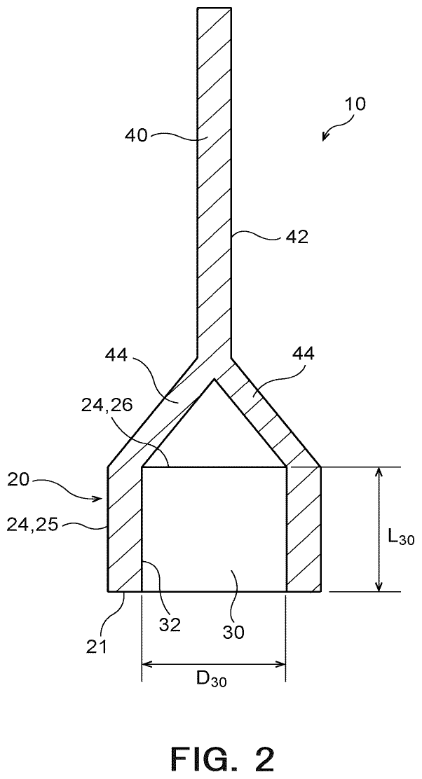

FIG. 1 is a perspective view illustrating an example of a sample collection device 10 according to an embodiment. FIG. 2 is a longitudinal cross section of the sample collection device 10.

The sample collection device 10 according to the embodiment includes a sample collection unit 20 and a grip 40. The sample collection unit 20 has a function to collect a sample 90 flowing out of a subject 70 and to retain the sample 90. The sample collection unit 20 has a contact surface 21, an outer surface 24, and a retaining space 30. In the embodiment, the sample collection unit 20 has a substantially cylindrical shape. The direction along which the central axis of the substantially cylindrical sample collection unit 20 extends (for example, the up and down direction in FIG. 2) may be called “axis direction” herein.

The contact surface 21 is configured to be capable of being in contact with a surface 72 of the subject 70. When the sample 90 is collected from the subject 70, the contact surface 21 is in contact with the surface 72 of the subject 70 as shown in FIGS. 4 to 6. In more detail, when the sample 90 is collected from the subject 70, the contact surface 21 is pressed on the surface 72 of the subject 70. In the embodiment, the contact surface 21 is flat. However, the contact surface 21 is not limited to be flat but may be curved or include a combination of a plurality of flat surfaces or a combination of a flat surface and a curved surface, for example. As will be described later, the retaining space 30 has an opening on the contact surface 21 as shown in FIGS. 1 and 2. In the embodiment, the contact surface 21 has a ring shape surrounding the opening of the retaining space 30. The shape of the contact surface 21 is not limited to the ring shape and may be other shapes such as an oval shape or a polygonal shape depending on the shape of the opening of the retaining space 30.

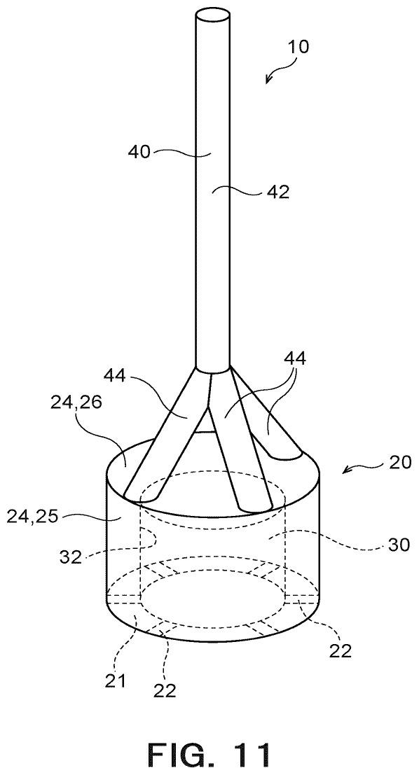

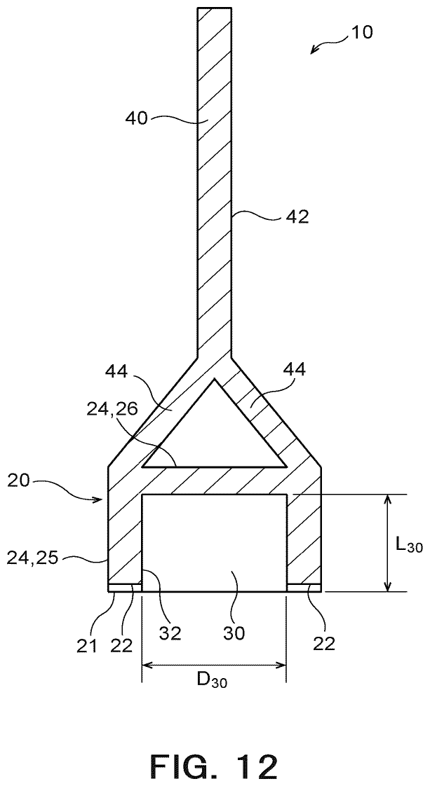

The outer surface 24 has a side surface 25 and an opposite surface 26. The opposite surface 26 is located opposite to the contact surface 21 in the sample collection unit 20. At least a part of the opposite surface 26 is located at an opposite side of the contact surface 21 in the axial direction of the sample collection unit 20. In the embodiment, the opposite surface 26 is a flat surface. However, the shape of the opposite surface 26 is not limited to be flat but may be curved or include a combination of a plurality of flat surfaces or a combination of a flat surface and a curved surface. As will be described later, in the example shown in FIGS. 1 and 2, the retaining space 30 also has an opening on the opposite surface 26. In the embodiment, the opposite surface 26 has a shape surrounding the opening of the retaining space 30. The shape of the opposite surface 26 is not limited the ring shape and may be other shapes such as an oval shape or a polygonal shape depending on the shape of the opening of the retaining space 30. As will be described later by referring to FIGS. 11 and 12, the retaining space 30 may not necessarily have an opening on the opposite surface 26.

The side surface 25 connects the contact surface 21 and the opposite surface 26, and is disposed on the outer side of the sample collection unit 20. In the embodiment, the side surface 25 has a cylindrical shape.

At least a part of the outer surface 24 may be hydrophobic. The term “hydrophobic” herein means that the angle of contact between a surface and water on the surface is 90 degrees or more. For example, the contact surface 21 may be hydrophobic. Preferably, the whole of the outer surface 24 is hydrophobic. If the outer surface 24 is hydrophobic, the sample 90 is prevented from adhering to the outer surface 24. As a result, when the sample 90 collected by the sample collection device 10 is processed in a subsequent step, it is possible to prevent the sample 90 from being additionally mixed into a processed material and causing a carry-over, since no sample 90 has adhered to the outer surface 24. The outer surface 24 may be formed of a hydrophobic material, or a material having been subjected to a hydrophobic treatment such as a silane coupling agent treatment, a fluorine plasma treatment, a treatment to apply a hydrophobic chemical, or the like.

The retaining space 30 is for retaining the sample 90. In other words, the retaining space 30 is configured to retain the sample 90. The retaining space 30 has an opening on the contact surface 21. In the example shown in FIGS. 1 and 2, the retaining space 30 also has an opening on the opposite surface 26. Thus, the retaining space 30 is formed as a through-hole penetrating the sample collection unit 20 in the axial direction. The retaining space 30 is substantially cylindrical. The opening on the contact surface 21 of the retaining space 30 is circular. The opening on the opposite surface 26 of the retaining space 30 is also circular. The retaining space 30 is surrounded by a wall 32. The retaining space 30 is defined by the wall 32, the opening on the contact surface 21 of the retaining space 30, and the opening on the opposite surface 26 of the retaining space 30. As will be described later by referring to FIGS. 11 and 12, it is not necessary that the retaining space 30 has the opening on the opposite surface 26.

The retaining space 30 holds therein a predetermined amount of the sample 90 by the surface tension of the sample 90. The sample 90 may be a body fluid of a human or an animal, and the body fluid may be blood or pus, for example. If the sample 90 is blood of a human, the diameter D30 of the retaining space 30 may be 0.1 mm or more and 100 mm or less, for example. If the diameter D30 is in the range of 0.1 mm to 100 mm, the blood may be appropriately retained in the retaining space 30 due to the surface tension of the blood. Preferably, the diameter D30 may be 0.1 mm or more and 10 mm or less.

The length L30 of the retaining space 30 is arbitrarily set depending on the amount of the sample 90 to be retained in the retaining space 30. The length L30 may be 0.5 mm or more and 100 mm or less, for example.

The volume (capacity) of the retaining space 30 may be arbitrarily set depending on the amount of the sample 90 to be retained in the retaining space 30. The volume of the retaining space 30 may be 0.05 μl or more and 10 ml or less, for example.

The wall 32 may be hydrophilic. The term “hydrophilic” herein means that the angle of contact between a surface and water on the surface is less than 90 degrees. If the wall 32 is hydrophilic, the sample 90 is more likely to be drawn into the retaining space 30 due to the surface tension of the sample 90. The wall 32 may be formed of a hydrophilic material, or a material having been subjected to a hydrophilic treatment such as plasma irradiation, wet processing using an acid or alkaline solution, dry processing using ultraviolet (UV) or ozone, application of a hydrophilic chemical, attachment of a hydrophilic film, or the like.

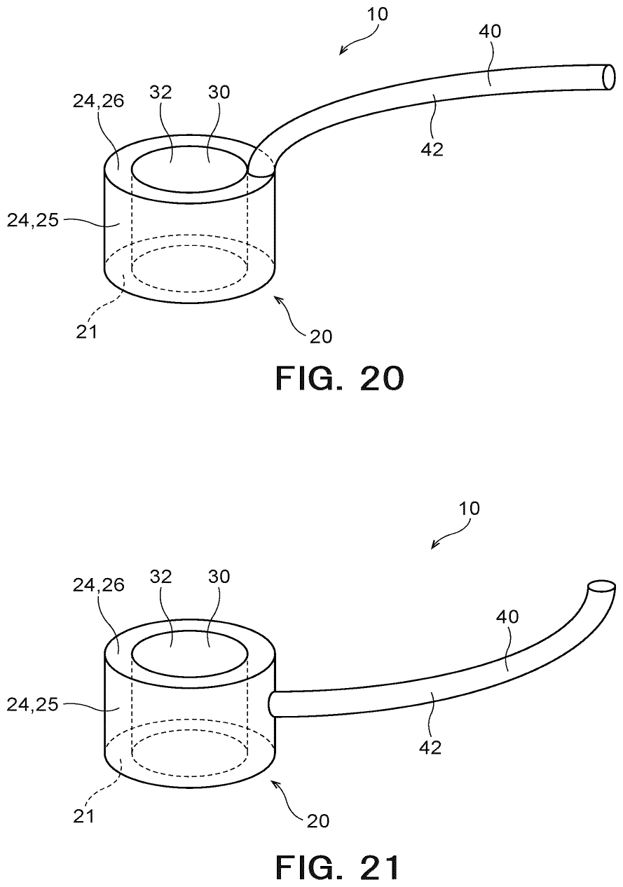

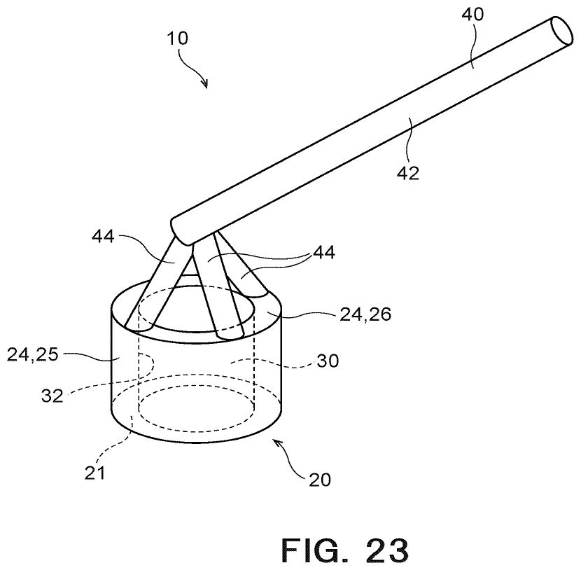

The grip 40 is intended to be held by a user. The user may hold the grip 40 to press the sample collection unit 20 to the surface 72 of the subject 70. The grip 40 is connected to the outer surface 24 of the sample collection unit 20. In the example shown in FIGS. 1 and 2, the grip 40 is connected to the opposite surface 26. The grip 40 has a leg 44, which connects a main body of the grip 40 to the sample collection unit 20. The main body of the grip 40 extends in parallel to the central axis of the sample collection unit 20. In particular, the main body of the grip 40 extends linearly along the central axis of the sample collection unit 20. One end of the leg 44 is connected to the main body of the grip 40, and the other end is connected to the sample collection unit 20. The grip 40 may have a plurality of legs 44. In the example shown in FIGS. 1 and 2, the grip 40 has four legs 44. The legs 44 may be arranged around the central axis of the sample collection unit 20 so as to have an equal angular pitch. If the grip 40 has four legs 44, the legs 44 may be arranged around the central axis of the sample collection unit 20 at an angular pitch of 90 degrees. The main body of the grip 40 does not necessarily extend in parallel to the central axis of the sample collection unit 20 (see FIG. 23). The main body of the grip 40 does not necessarily extend linearly, either. For example, the main body of the grip 40 may have a curved shape or an angular shape (see FIGS. 20 and 21).

The grip 40 has a surface 42. At least a part of the surface 42 may be hydrophobic. Preferably, the whole of the surface 42 is hydrophobic. If the surface 42 is hydrophobic, the sample 90 is prevented from adhering to the surface 42. As a result, when the sample 90 collected by the sample collection device 10 is processed in a subsequent step, it is possible to prevent the sample 90 from being additionally mixed into a processed material and causing a carry-over, since no sample 90 has adhered to the surface 42. The surface 42 may be formed of a hydrophobic material, or a material having been subjected to a hydrophobic treatment such as a silane coupling agent treatment, a fluorine plasma treatment, a treatment to apply a hydrophobic chemical, or the like.

The sample collection device 10 may be formed of a hard material. The hard material may be a highly rigid material. The hard material may be at least one of ceramics, metals, glass, and resins. The materials of the sample collection unit 20 and the grip 40 may be the same or different. If the grip 40 is formed of a hard material, when the user holding the grip 40 presses the sample collection unit 20 to the surface 72 of the subject 70, the grip 40 is prevented from deforming. As a result, it is possible to appropriately press the sample collection unit 20 to the surface 72 of the subject 70.

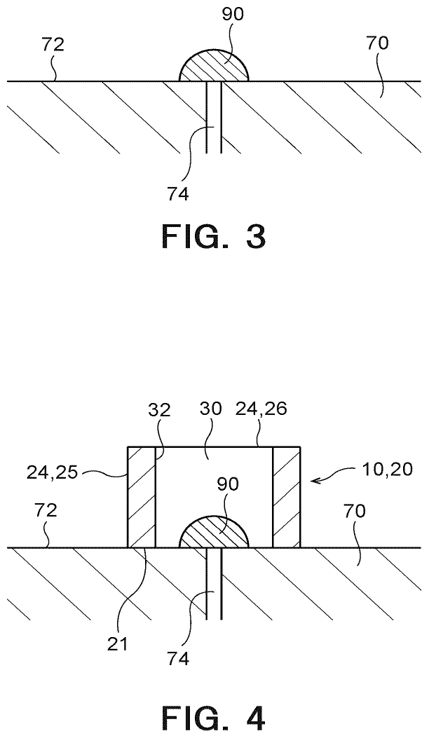

Next, an example of a method of collecting the sample 90 using the sample collection device 10 will be described with reference to FIGS. 3 to 7. In this example, the sample 90 is blood.

First, the surface 72 of a finger, for example, of the subject 70 is punctured using a lancet, for example. As shown in FIG. 3, a sample outflow hole 74 is formed at the punctured portion of the surface 72. Blood (sample 90) flows out of the sample outflow hole 74.

The user holds the grip 40 to bring the contact surface 21 of the sample collection unit 20 into contact with the surface 72 around the sample outflow hole 74 as shown in FIG. 4. As this time, the retaining space 30 of the sample collection unit 20 is placed over the sample outflow hole 74. In FIGS. 4 to 7, the grip 40 is not illustrated.

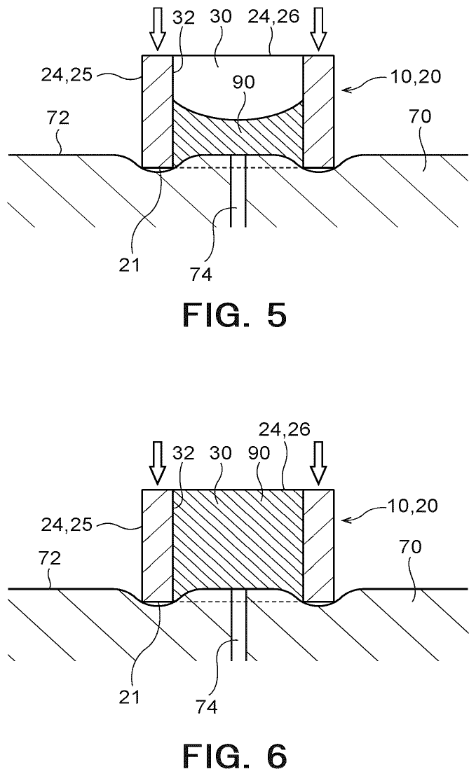

Next, the user presses the contact surface 21 of the sample collection unit 20 to the surface 72 of the subject 70 around the sample outflow hole 74. As a result, the contact surface 21 pushes the portion around the sample outflow hole 74 to retract, thereby causing more blood to flow from the sample outflow hole 74 into the retaining space 30, as shown in FIG. 5. Preferably the wall 32 surrounding the retaining space 30 is hydrophilic to improve the fluid surface keeping force due to the surface tension of the blood.

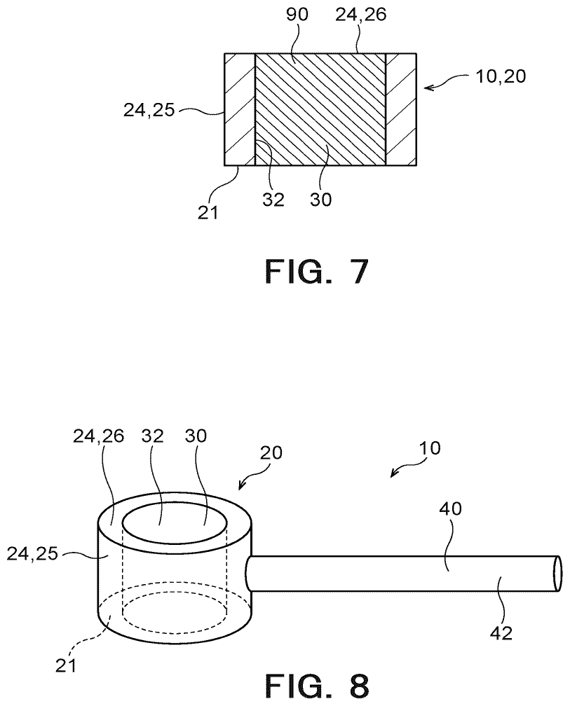

The blood flowing out of the sample outflow hole 74 into the retaining space 30 fills the entire part of the retaining space 30 as shown in FIG. 6. If the contact surface 21 of the sample collection unit 20 is removed from the surface 72 of the subject 70 in this state, a predetermined amount of blood is left in the retaining space 30 as shown in FIG. 7. It is not necessary that the entire part of the retaining space 30 is filled with the sample 90. For example, a part of the wall 32 surrounding the retaining space 30 may be hydrophilic or hydrophobic so as to meet the requirement of setting the predetermined amount of the sample 90 retained in the retaining space 30 when a part of the retaining space 30 is filled with the sample 90.

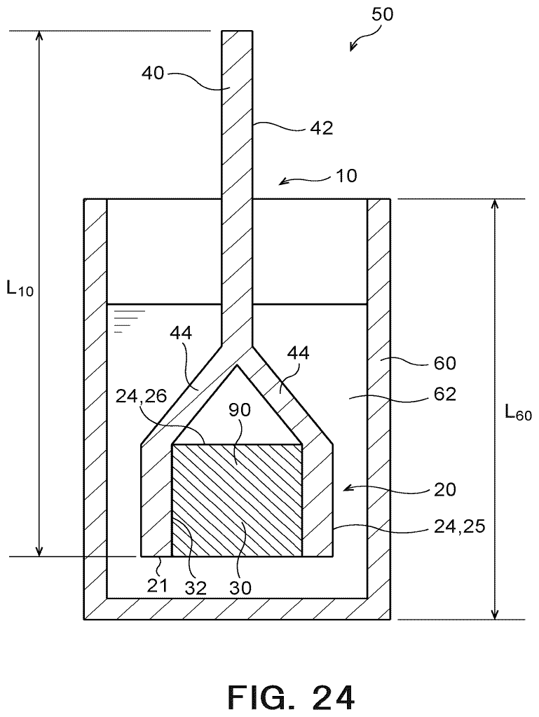

Thereafter, the sample 90 retained in the retaining space 30 may be mixed into a processing solution 62 stored in a sample processing container 60, which will be described later (see FIG. 24).

The sample collection device 10 according to the embodiment includes the sample collection unit 20 having the contact surface 21 that may be in contact with the surface 72 of the subject 70 on which the sample outflow hole 74 is formed, the retaining space 30 having the opening on the contact surface 21, and the outer surface 24. The predetermined amount of sample 90 obtained from the sample 90 flowing out of the sample outflow hole 74 is retained in the retaining space 30 due to the surface tension of the sample 90 when the contact surface 21 is pressed to the surface 72 around the sample outflow hole 74.

According to the sample collection device 10, it is possible to cause the sample 90 to flow out of the sample outflow hole 74 by pressing the contact surface 21 of the sample collection unit 20 to the surface 72 round the sample outflow hole 74, and to retain a predetermined amount of sample 90 obtained from the sample 90 flowing out of the sample outflow hole 74 due to the surface tension of the sample 90. As a result, it is possible to easily collect the predetermined amount of sample 90 from the subject 70. Therefore, in a process of POCT, a predetermined amount of sample 90 may be collected easily and at a low cost. Furthermore, no special knowledge or technique is needed to collect a predetermined amount of sample 90, and the squeezing of the sample 90 out of the sample outflow hole 74 and the collecting of a predetermined amount of sample 90 may be performed at the same time.

In the sample collection device 10 according to the embodiment, the predetermined amount is determined by at least one of the volume of the retaining space 30, the shape of the retaining space 30, the material of the collection unit 20, and the chemical property of the wall 32 surrounding the retaining space 30.

The chemical property of the wall 32 surrounding the retaining space 30 is, for example, whether the wall 32 is hydrophilic or hydrophobic. Thus, the sample collection device 10 is capable of measuring the predetermined amount of sample 90 with a simple configuration.

The sample collection device 10 according to the embodiment includes the grip 40 connected to the outer surface 24.

According to the sample collection device 10, the user may hold the grip 40 to press the contact surface 21 of the sample collection unit 20 to the surface 72 of the subject 70. Thus, it is possible to easily press the contact surface 21 to the surface 72 of the subject 70.

The grip 40 is formed of a hard material in the sample collection device 10 according to the embodiment.

In the sample collection device 10, if the grip 40 is formed of a hard material, the grip 40 is prevented from deforming when the user holding the grip 40 presses the sample collection unit 20 to the surface 72 of the subject 70. As a result, the sample collection unit 20 may be appropriately pressed against the surface 72 of the subject 70.

In the sample collection device 10 according to the embodiment, the wall 32 surrounding the retaining space 30 is hydrophilic.

According to the sample collection device 10, the sample 90 may be easily drawn into the retaining space 30 due to the surface tension of the sample 90.

In the sample collection device 10 according to the embodiment, at least a part of the contact surface 21 and/or at least a part of the outer surface 24 are/is hydrophobic.

In the sample collection device 10 according to the embodiment, at least a part of the surface 42 of the grip 40 is hydrophobic.

If at least a part of the contact surface 21 is hydrophobic, only the part of the retaining space 30 may retain the sample 90. As a result, regardless of the volume of the retaining space 30, it is possible to retain a predetermined amount of the sample 90 in the retaining space 30.

If at least a part of the outer surface 24 and/or at least a part of the surface 42 of the grip 40 are/is hydrophobic, the sample 90 is prevented from adhering to the outer surface 24 and/or the surface 42. As a result, when the sample 90 collected by the sample collection device 10 is processed in a subsequent step, it is possible to prevent the sample 90 from being additionally mixed into a processed material and causing a carry-over, since no sample 90 has adhered to the outer surface 24 and/or the surface 42.

The embodiment described above may be modified in various ways. Examples of the modification will be described below with reference to the drawings. In the following descriptions and the drawings used in the descriptions, elements having substantially the same structures have the same numerical symbols, and the explanations of such elements are not repeated.

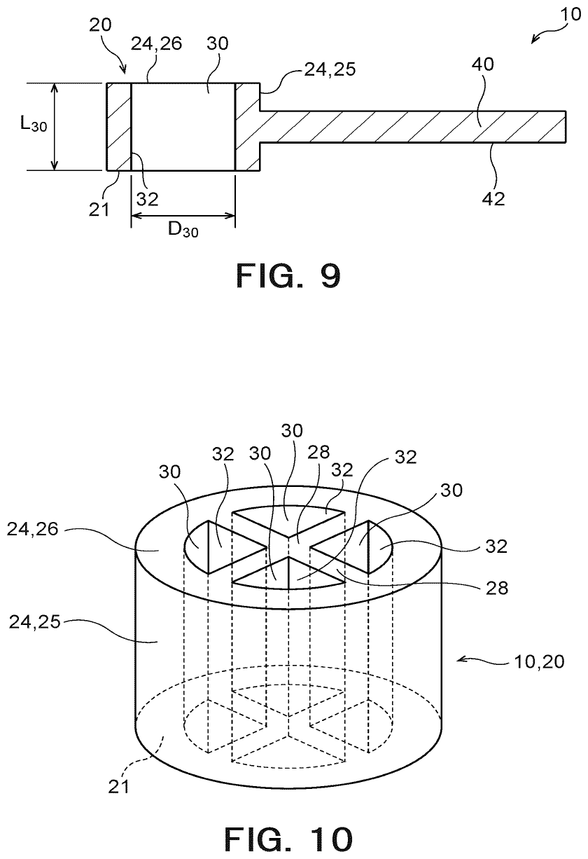

FIG. 8 is a perspective view of a modification of the sample collection device 10. FIG. 9 is a longitudinal cross section of the sample collection device 10 shown in FIG. 8.

In the example shown in FIGS. 8 and 9, the grip 40 is connected to the side surface 25 of the outer surface 24. The grip 40 extends in the direction crossing the central axis of the sample collection unit 20. Although the grip 40 does not have legs 44 in the shown example, the configuration of the grip 40 is not limited to this example, and the grip 40 may have one or more legs 44.

The sample collection device 10 including such a grip 40 may also be able to cause the sample 90 to flow out of the sample outflow hole 74 by pressing the contact surface 21 of the sample collection unit 20 to the surface 72 around the sample outflow hole 74, and to cause a predetermined amount of the sample 90 obtained from the sample 90 flowing out of the sample outflow hole 74 to be retained in the retaining space 30 using the surface tension of the sample 90.

FIG. 10 is a perspective view of another modification of the sample collection device 10. In the example shown in FIG. 10, the sample collection unit 20 has a plurality of retaining spaces 30. The retaining spaces 30 of the sample collection unit 20 are disposed around the central axis of the sample collection unit 20. The retaining spaces 30 may be disposed to have the same angular pitch around the central axis of the sample collection unit 20. As shown in FIG. 10, if the sample collection unit 20 has four retaining spaces 30, the four retaining spaces 30 may be disposed around the central axis of the sample collection unit 20 at an angular pitch of 90 degrees. Adjacent two retaining spaces 30 are partitioned by a partition wall 28.

In the sample collection device 10 having such retaining spaces 30, since the cross-sectional area of each retaining space 30 becomes small, it is easier to draw the sample 90 into each retaining space 30 due to so-called capillarity. The grip 40 having been described with reference to FIGS. 1 and 2 and/or the grip 40 having been described with reference to FIGS. 8 and 9 may be connected to the sample collection unit 20 shown in FIG. 10.

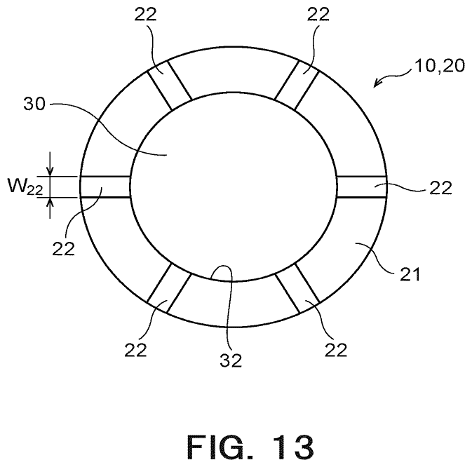

FIG. 11 is a perspective view of a further modification of the sample collection device 10. FIG. 12 is a longitudinal cross section of the sample collection device 10 shown in FIG. 11. FIG. 13 is a diagram showing the sample collection device 10 of FIG. 11 from the contact surface side.

In the sample collection device 10 according to this modification, the retaining space 30 does not have an opening on the opposite surface 26. In other words, the retaining space 30 in this modification is open only to the contact surface 21. The sample collection device 10 having such a retaining space 30 is also capable of causing the sample 90 to flow out of the sample outflow hole 74 by pressing the contact surface 21 of the sample collection unit 20 to the surface 72 around the sample outflow hole 74, and causing a predetermined amount of the sample 90 obtained from the sample 90 flowing out of the sample outflow hole 74 to be retained in the retaining space 30 due to the surface tension of the sample 90.

In this modification, the contact surface 21 has one or more grooves 22. Each groove 22 extends to connect the retaining space 30 and the side surface 25. Each groove 22 may serve as vents through which air flows out of the retaining space 30 when the sample 90 flows into the retaining space 30. Therefore, the risk may be prevented that the opening of the retaining space 30 may be closed by the surface 72 of the subject 70 and the air in the retaining space 30 cannot flow out of the retaining space 30 when the sample 90 flows into the retaining space 30. As a result, the sample 90 may smoothly flow into the retaining space 30.

The contact surface 21 may have a plurality of grooves 22, which may be arranged at a constant angular pitch around the central axis of the sample collection unit 20. If the contact surface 21 has six grooves 22 as shown in FIG. 13, the six grooves 22 may be disposed around the central axis of the sample collection unit 20 at an angular pitch of 60 degrees. The width W22 of the grooves 22 may be from a few μm to a few cm, for example.

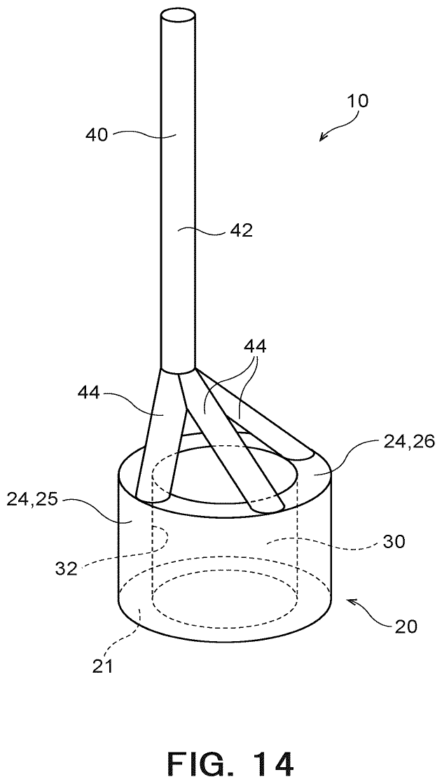

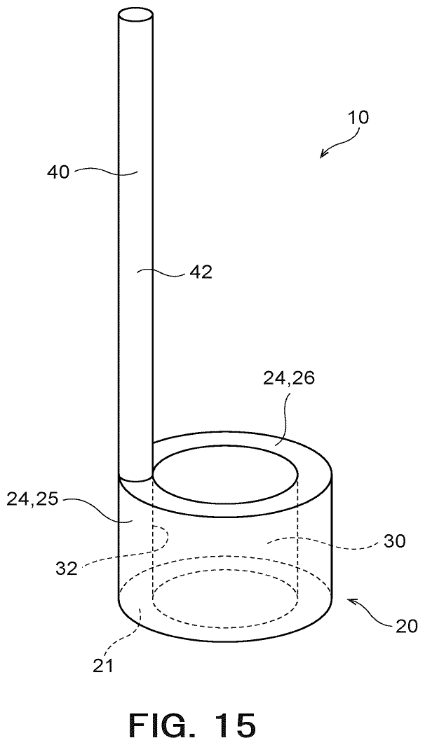

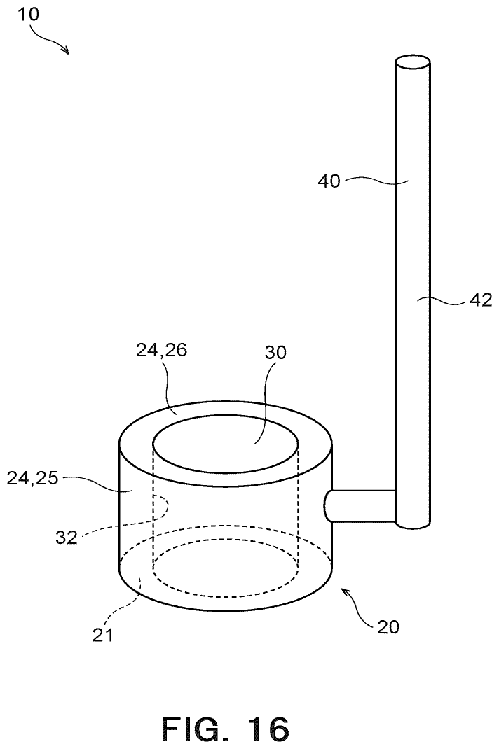

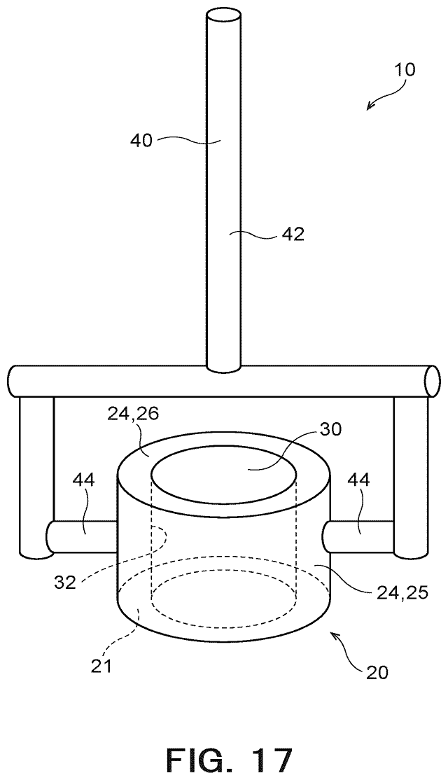

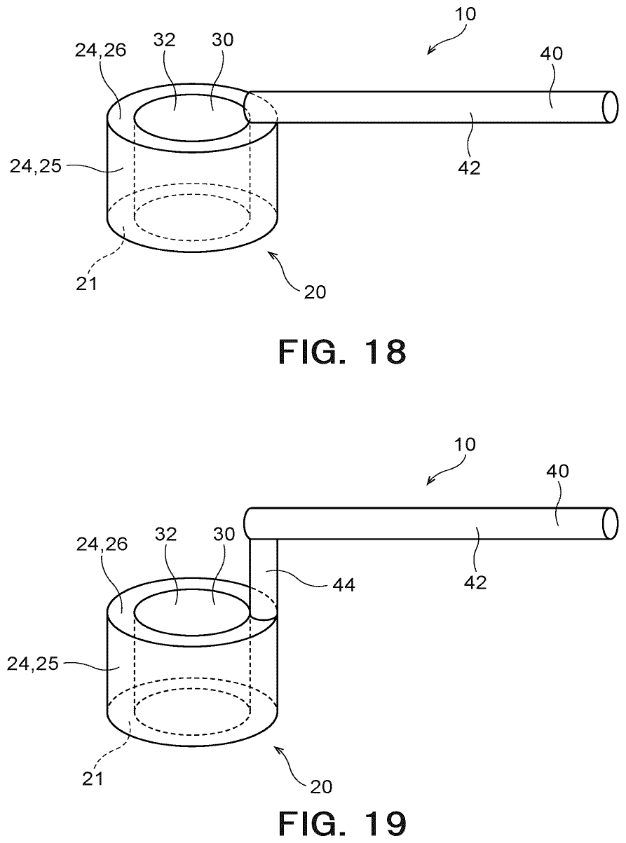

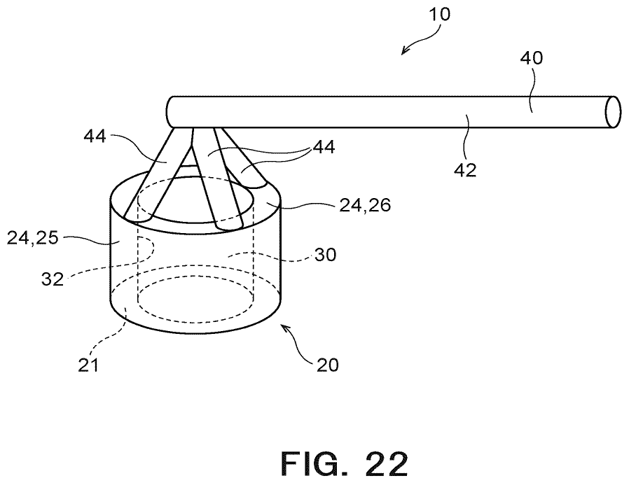

FIGS. 14 to 23 show still further modifications of the sample collection device 10. As shown in FIG. 14, the grip 40 may extend in parallel to and offset from the central axis of the sample collection unit 20. As shown in FIG. 15, the grip 40 may be disposed directly on the opposite surface 26 so as to extend in parallel to the central axis of the sample collection unit 20. As shown in FIG. 16, the grip 40 may be attached to the side surface 25 and the main body of the grip 40 may extend in parallel to the central axis of the sample collection unit 20. As shown in FIG. 17, the grip 40 may be attached to opposite sides of the side surface 25 and the main body of the grip 40 may extend along the central axis of the sample collection unit 20. As shown in FIG. 18, the grip 40 may be attached to the opposite surface 26 so as to extend in a direction crossing the central axis of the sample collection unit 20, in particular in a direction perpendicular to the central axis of the sample collection unit 20. As shown in FIG. 19, the grip 40 may have a leg 44 attached to the opposite surface 26 and a main body extending in a direction crossing the central axis of the sample collection unit 20, in particular a direction perpendicular to the central axis of the sample collection unit 20. As shown in FIG. 20, the grip 40 may have a curved shape extending from the opposite surface 26. As shown in FIG. 21, the grip 40 may have a curved shape extending from the side surface 25. As shown in FIG. 22, the grip 40 may have a plurality of legs 44 attached to the opposite surface 26 and a main body extending in a direction crossing the central axis of the sample collection unit 20, in particular in a direction perpendicular to the central axis of the sample collection unit 20. As shown in FIG. 23, the grip 40 may have a plurality of legs 44 attached to the opposite surface 26 and a main body extending in a direction crossing the central axis of the sample collection unit 20, in particular a direction that is inclined relative to the central axis of the sample collection unit 20.

In the examples shown in FIGS. 1, 11, and 14 to 17, the main body of the grip 40 extends in parallel to the central axis of the sample collection unit 20. Therefore, when the sample collection unit 20 is pressed to the surface 72 of the subject 70, the force applied by the user may be conveyed to the sample collection unit 20 easily. In the examples shown in FIGS. 15, 16, and 18 to 21, the grip 40 is not located above the retaining space 30. Therefore, the surface 72 of the subject 70 may be more easily confirmed visually through the retaining space 30. In the examples shown in FIGS. 14 to 23, the retaining space 30 does not necessarily have an opening on the opposite surface 26, like the examples shown in FIGS. 11 and 12.

FIG. 24 is a longitudinal cross section of a sample processing device 50.

In the example shown in FIG. 24, the sample processing device 50 includes a sample collection device 10 and a sample processing container 60. The sample processing container 60 is used for the processing of the sample 90. A predetermined amount of processing solution 62 is contained in the sample processing container 60. The processing solution 62 is a diluent, for example. A sample having a predetermined concentration may be obtained by diluting the predetermined amount of sample 90 stored in the sample collection device 10 with the processing solution 62 contained in the sample processing container 60.

In the example shown in FIG. 24, the sample collection unit 20 has a size that is suitable for being housed in the sample processing container 60. This allows the sample collection unit 20 to be immersed in the processing solution 62 contained in the sample processing container 60. Thus, it is possible to easily mix the sample 90 stored in the sample collection device 10 with the processing solution 62 contained in the sample processing container 60.

Furthermore, in the example shown in FIG. 24, the length L10 of the sample collection device 10 is longer than the length L60 of the sample processing container 60. Therefore, the user may insert the sample collection unit 20 into the sample processing container 60 while holding the grip 40. Thus, it is possible to more easily mix the sample 90 stored in the sample collection device 10 with the processing solution 62 in the sample processing container 60.

### First Experiment

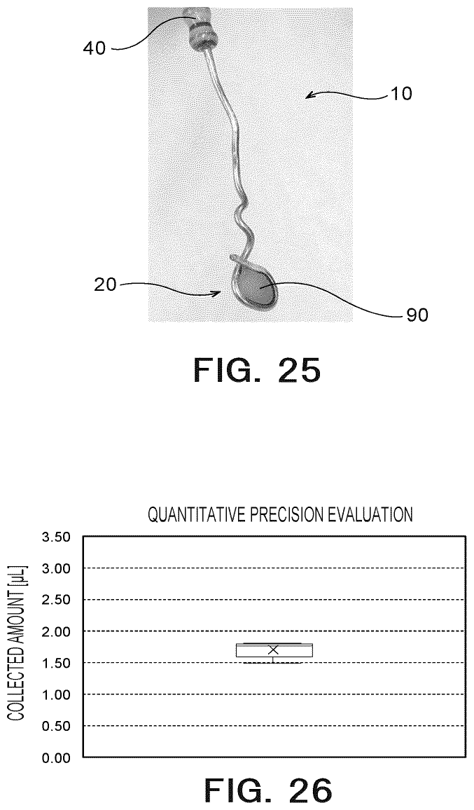

Next, a first experiment performed by the inventors of the present invention will be described with reference to FIGS. 25 and 26. FIG. 25 is a photograph of the sample collection device 10 used in the first experiment. FIG. 26 shows an evaluation of quantitative precision of the sample collected by using the sample collection device 10 shown in FIG. 25. The inside diameter of the sample collection unit 20 of the sample collection device 10 used in the first experiment was about 0.3 cm, and the thickness was about 0.05 cm. The sample 90 was bovine blood serum.

First, the sample collection unit 20 is immersed into the sample 90 contained in a 1.5 mL tube to store the sample 90 in the retaining space 30. Thereafter, the sample collection unit 20 is immersed into a dilution solvent (processing solution 62) contained in another 1.5 ml tube (sample processing container 60) to mix the sample 90 with the dilution solvent by agitation to obtain a diluted sample. Using this sample, a 280 nm absorbance value was measured. From a calibration curve showing the relationship between 280 nm absorbance value and dilution ratio that had been obtained in advance, the dilution ratio and the collected amount of the sample were calculated. This was repeated six times. Thus, the absorbance value was measured, and the dilution ratio and the collected amount were calculated for six samples. FIG. 26 shows the result represented by a box plot indicating the dispersion of collected amounts calculated for the six samples.

The average collected amount for the six collections of the sample 90 was 1.7 μL, and the SD value (standard deviation) was 0.112. It is considered from this result that the collected amount of the sample 90 was constant and no carry-over occurred, and the quantitative precision was considerably high. Furthermore, since all of the collected sample 90 may be mixed into the dilution solvent, the risk of carry-over of the sample 90 may be low. This is considered to lead not only to the improvement of quantitative precision but also to the reduction in risk of biohazard and/or contamination.

### Second Experiment

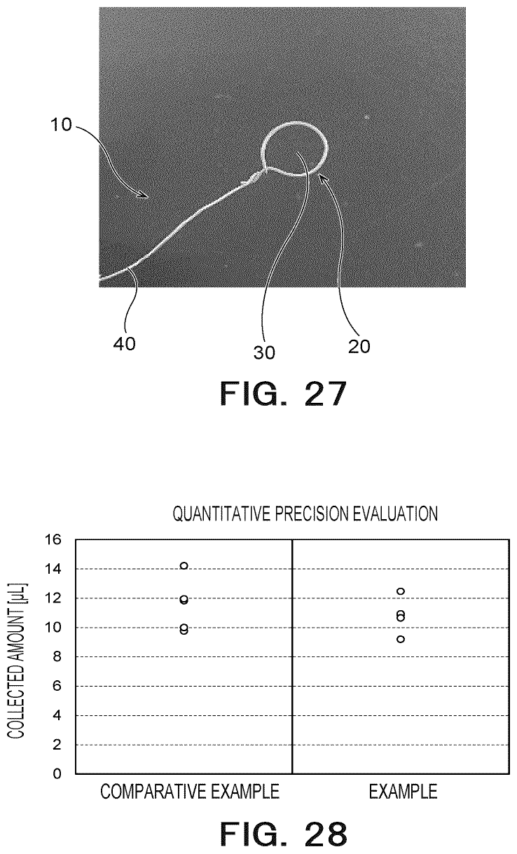

Next, a second experiment performed by the inventors will be described with reference to FIGS. 27 and 28. FIG. 27 is a photograph of the sample collection device 10 used in the second experiment. FIG. 28 shows an evaluation of quantitative precision of the sample 90 collected in the second experiment.

The retaining space 30 of the sample collection unit 20 included in the sample collection device 10 used for the second experiment was in a substantially elliptical planar shape having a major axis of about 5 mm and a minor axis of about 4 mm, and a thickness of about 0.35 mm. The sample 90 was 100% glycerol imitating viscous sample such as blood. The 100% glycerol was cooled by ice and the viscosity thereof was adjusted to be from 5000 cp to 10000 cp. Then the 100% glycerol was mixed with a red pigment to make the sample 90. An experiment was performed as an example using the sample collection device 10 shown in FIG. 27. As a comparative example, an experiment was performed using a commercially available micropipette manufactured by Eppendorf SE (Eppendorf Reference® 2).

First, the sample 90 contained in a 1.5 ml tube was collected by using the sample collection device 10 (Example) or the micropipette (Comparative Example). Next, the collected sample 90 was added to water (processing solution 62) contained in another 1.5 ml tube (sample processing container 60) and agitated to be diluted to form the sample. Since the sample retainability of the sample collection device 10 used in the second experiment was calculated to be 11 μL from the relationship between the 500 nm absorbance value and the sample collection amount measured in advance, the amount of the sample 90 collected by the micropipette was also 11 μL. For each sample, the 500 nm absorbance value was measured five times. The collection amount of each sample was calculated from the relationship between the 500 nm absorbance value and the sample collection amount measured in advance.

In Comparative Example, the average value of the amount of the sample 90 collected by using the micropipette was 11.5 μL, and the standard deviation (SD) was 1.60. In Example, the average value of the amount of the sample 90 collected by using the sample collection device 10 shown in FIG. 27 was 10.8 μL, and the standard deviation (SD) was 1.03. Thus, the standard deviation (SD) in the case where the sample collection device 10 was used in Example was smaller than the standard deviation (SD) in the case where the micropipette was used in Comparative Example. It can be understood from this result that even if the sample 90 has a high viscosity like this experiment, the fluctuations in the amount of the sample 90 collected by the sample collection device 10 are small, which leads to the improvement in quantitative precision (see FIG. 28).

If the sample 90 having a high viscosity is dispensed by using the micropipette according to Comparative Example, the time needed for the dispensing is relatively long due to the high flow path resistance caused by the high viscosity. On the other hand, if the sample 90 is dispensed by using the sample collection device 10 according to Example, it is possible to prevent the dispense time from being elongated due to the high viscosity. For example, the work time needed for dispensing the sample 90 using the sample collection device 10 according to Example may be shortened to about two-thirds of the time needed for dispensing the sample 90 using the micropipette according to Comparative Example. Thus, the sample collection device 10 according to Example has an advantageous effect to considerably decrease the working time.

While certain embodiments and their modifications have been described, these embodiments and modifications have been presented by way of example only, and are not intended to limit the scope of the inventions. Indeed, a variety of other forms such as omissions, substitutions, changes and combinations may be made in the embodiments and modifications without departing from the spirit of the inventions. Such embodiments and modifications are intended to be covered by the scope and spirit of the inventions as well as the claimed inventions and their equivalents.

### Subjoinder 1

A sample collection device comprising:
a sample collection unit having a contact surface capable of being in contact with a surface having a sample outflow hole of a subject, a retaining space having an opening on the contact surface, and an outer surface,
the sample collection device being configured to retain in the retaining space a predetermined amount of a sample due to surface tension of the sample, the predetermined amount of the sample being obtained from the sample flowing out of the sample outflow hole when the contact surface is pressed to the surface around the sample outflow hole.

### Subjoinder 2

The predetermined amount may be determined by at least one of a volume of the retaining space, a shape of the retaining space, a material of the sample collection unit, and a chemical property of a wall defining the retaining space.

### Subjoinder 3

The sample collection device may further comprise a grip connected to the outer surface.

### Subjoinder 4

The grip may be formed of a hard material.

### Subjoinder 5

A wall defining the retaining space may be hydrophilic.

### Subjoinder 6

At least a part of the contact surface and/or at least a part of the outer surface may be hydrophobic.

### Subjoinder 7

At least a part of a surface of the grip may be hydrophobic.

### Subjoinder 8

A sample processing device may comprise:
the sample collection device; and
a sample processing container used for processing the sample,
the sample collection unit having a size suitable to be housed in the sample processing container.

### Subjoinder 9

A sample processing device may comprise:
the sample collection device; and
a sample processing container used for processing the sample,
a length of the sample collection device being longer than a length of the sample processing container.

1. A sample collection device comprising:
a sample collection unit having a contact surface capable of being in contact with a surface having a sample outflow hole of a subject, a retaining space having an opening on the contact surface, and an outer surface,
the sample collection device being configured to retain in the retaining space a predetermined amount of a sample due to surface tension of the sample, the predetermined amount of the sample being obtained from the sample flowing out of the sample outflow hole when the contact surface is pressed to the surface around the sample outflow hole.

2. The sample collection device according to claim 1, wherein the predetermined amount is determined by at least one of a volume of the retaining space, a shape of the retaining space, a material of the sample collection unit, and a chemical property of a wall defining the retaining space.

3. The sample collection device according to claim 1, further comprising a grip connected to the outer surface.

4. The sample collection device according to claim 3, wherein the grip is formed of a hard material.

5. The sample collection device according to claim 1, wherein a wall defining the retaining space is hydrophilic.

6. The sample collection device according to claim 1, wherein at least a part of the contact surface and/or at least a part of the outer surface are/is hydrophobic.

7. The sample collection device according to claim 3, wherein at least a part of a surface of the grip is hydrophobic.

8. A sample processing device comprising:
the sample collection device according to claim 1; and
a sample processing container used for processing the sample,
the sample collection unit having a size suitable to be housed in the sample processing container.

9. A sample processing device comprising:
the sample collection device according to claim 3; and
a sample processing container used for processing the sample,
a length of the sample collection device being longer than a length of the sample processing container.
